(19) United States
(12) Patent Application Publication
(10) Pub. No.: US 2019/0072534 A1
(43) Pub. Date: Mar. 7, 2019

(54) STOPPED FLOW WITH PULSED INJECTION TECHNIQUE FOR TOTAL ORGANIC CARBON ANALYZER (TOCA) USING HIGH TEMPERATURE COMBUSTION

(71) Applicant: O.I. Corporation, College Station, TX (US)

(72) Inventors: Richard K. SIMON, College Station, TX (US); Noel C. BAUMAN, College Station, TX (US); Nathan C. RAWLS, Somerville, TX (US); Karl WILLIAMS, Bryan, TX (US)

(21) Appl. No.: 16/057,372

(22) Filed: Aug. 7, 2018

Related U.S. Application Data
(60) Provisional application No. 62/541,916, filed on Aug. 7, 2017.

(51) Int. Cl. G01N 33/18; B01L 7/00

(52) U.S. Cl. CPC G01N 33/1846; G01N 2001/002; B01L 7/525

(57) ABSTRACT

According to some embodiments, the present invention may include, or take the form of, a total organic carbon analyzer, featuring an injector, a reactor, condensation components and two three-way valves. The injector may be configured to provide a sample. The reactor may be configured to vaporize the sample received. The condensation components may be configured to condense and trap the sample vaporized by the reactor. The two three-way valves may be arranged between the reactor and the condensation components and configured to allow flow to either bypass or pass through the reactor and the condensation components, while in the bypass mode, the sample being injected at an appropriate rate so as to allow the sample to condense at or near the same rate as the sample is being injected.

### CROSS-REFERENCE TO RELATED APPLICATIONS

This application claims benefit to provisional patent application Ser. No. 62/541,916 (911-027.3-1/N-OIC-0020US), filed 7 Aug. 2017, which is incorporated by reference in its entirety.

This application is also related to application Ser. No. 15/807,159, filed 8 Nov. 2017, entitled “Smart slide,” which is both hereby incorporated by reference in its entirety.

### BACKGROUND OF THE INVENTION

### 1. Field of Invention

This invention relates to a technique for detecting carbon in a sample; and more particular to a total organic carbon analyzer for detecting the same.

### 2. Description of Related Art

Injection of a known volume of aqueous samples into a combustion TOCA results in the oxidation of the carbon in the sample to carbon dioxide. The TOCA consists of a sample injection mechanism, a heated reactor (constant temperature, nominally 680° C. to 900° C.), a catalytic bed, a condensation mechanism, a drying mechanism, filters for removal of chlorine, water, and particulates, and a detector for quantitation of the carbon in the sample (measured as carbon dioxide).

Conventionally, a TOCA utilizes a reaction chamber into which a sample is introduced, which provides the thermal energy to vaporize the sample, and heat the resulting vapor to the required catalytic temperature. In conventional operation, the TOCA provides a continuous flow of oxygen or air into the reactor chamber which contains a catalytic bed held at the required temperature for conversion of the carbon in the sample to carbon dioxide. The outlet from the reaction chamber is into a condensation and/or water removal chamber. The gas stream then is chemically filtered and the carbon dioxide is detected by a non-dispersive infra-red detector (NDIR) that is specifically designed for the detection of carbon dioxide.

Gas pressure is generated by the both the gas flow devices and the expansion pulse due to vaporization of the aqueous sample during injection of the analyte. Gas flow devices can consist of mass flow controllers, pressure regulators with frit or other gas flow controllers and can be mechanical or electronically controlled. Conventionally, the gas flow is always on, always passes through the reactor, and provides the primary motive force for the passage of the gas, vaporized sample, and reaction products through the system. Upon an injection of sample, a thermal gradient across a catalyst bed is created due to the energy required to vaporize the injected sample, and heat the resulting steam to the reactor temperature. During the vaporization process, the aqueous sample rapidly heats and converts the water to steam. The conversion of the aqueous sample to steam creates a pressure pulse, and also cools the leading section of the catalyst bed. Sufficient catalyst mass is therefore required to ensure complete oxidation of the sample during the unstopped flow across the catalyst bed. Since aqueous samples expand dramatically upon vaporization, the reactor design has to be capable of withstanding the injection generated pressure pulse. At the same time, sample will be adsorbed on the cooled catalyst until the reactor heats up sufficiently to fully convert the water to steam. The sample is finally oxidized as it moves across the heated catalytic bed. The net effect is often a broad, multi-modal peak shape being detected by the NDIR, requiring an extended analysis time, making quantitation difficult for large injection volumes.

Below is also a description of some other known technique disclosed in associated patents.

In view of this, there is a need in the art for a better way to detect carbon in a sample, e.g., using a total organic carbon analyzer.

### SUMMARY OF THE INVENTION

According to some embodiments, the present invention may include, or take the form of, a total organic carbon analyzer, featuring an injector, a reactor, condensation components and two three-way valves.

The injector may be configured to provide a sample.

The reactor may be configured to vaporize the sample received.

The condensation components may be configured to condense and trap the sample vaporized by the reactor.

The two three-way valves may be arranged between the reactor and the condensation components and configured to allow flow to either bypass or pass through the reactor and the condensation components, while in the bypass mode, the sample being injected at an appropriate rate so as to allow the sample to condense at or near the same rate as the sample is being injected.

The total organic carbon analyzer may include one or more of the following additional features:

The two three-way valves may include:
a stop flow valve V1 having a port C, a normally open port NO and a normally closed port NC; and a flow valve V2 having a corresponding port C, a corresponding normally open port NO and a normally closed port NC.

The condensation components may include a condensate trap and a total inorganic carbon (TIC) and condensate trap. The normally open port NO of the stop flow valve V1 may be coupled to a port of the reactor. The normally closed port NC of the stop flow valve V1 may be coupled to the corresponding normally closed port of the flow valve V2. The corresponding normally opening port NO of the flow valve V2 may be coupled to and receives condensate trap CO2 gas from the condensate trap. The corresponding port C of the flow valve V2 may be coupled to the TIC and condensate trap to provide the condensate trap CO2 gas from the condensate trap to the TIC and condensate trap.

The condensation components may include a primary condenser coupled between the reactor and the condensate trap and configured to receive reactor CO2 gas from the reactor and provide primary condenser CO2 gas to the condensate trap.

The TIC and condensate trap may be configured to receive the sample.

The total organic carbon analyzer may include a check valve coupled between the normally open port NO of the stop flow valve V1 and the port of the reactor.

The total organic carbon analyzer may include a humidifier coupled to provide humidifier gas to the port C of the stop flow valve V1.

The total organic carbon analyzer may include a nafion tube immersed in water and coupled to provide humidifier gas to the port C of the stop flow valve V1.

The total organic carbon analyzer may include a non-dispersive infra-red detector (NDIR) configured to receive the TIC and condensate CO2 gas, detect of the carbon dioxide contained therein and provide NDIR signaling containing information about the same. Alternatively, the total organic carbon analyzer may include a mass spectrometer, an ion conductivity sensor, a cavity ring down spectrometer (isotope ratio) that is specific for carbon dioxide, or a Fourier-transform infrared (FTIR) spectrometers.

The total organic carbon analyzer may include a combination of a pressure regulator and a mass flow controller configured to regulate gas flow.

The total organic carbon analyzer may include an electronic flow control, and/or electronic pressure regulation configured to regulate gas flow.

Embodiments are envisioned, and the scope of the invention is intended to include, a total organic carbon analyzer, featuring an injector, a reactor, condensation components and two three-way valves.

Consistent with that set forth above, the injector may be configured to provide a sample; the reactor may be configured to vaporize the sample received; and the condensation components may be configured to condense and trap the sample vaporized by the reactor. In this embodiment, the multi-way valve arrangement may be arranged between the reactor and the condensation components and configured to allow flow to either bypass or pass through the reactor and the condensation components, while in the bypass mode, the sample being injected at an appropriate rate so as to allow the sample to condense at or near the same rate as the sample is being injected. The multi-way valve arrangement may include the two three-way valves, as set forth herein. Alternatively, the multi-way valve arrangement may include a single 4 port valve.

### BRIEF DESCRIPTION OF THE DRAWING

The patent or patent application contains at least one drawing executed in color. Copies of this patent or patent application publication with color drawing(s) will be provided by the Patent Office upon request and payment of the necessary fee.

The drawing, which are not necessarily drawn to scale, includes FIGS. 1-12, as follows:

FIG. 1 is a plumbing diagram of a total organic carbon analyzer, according to some embodiments of the present invention.

FIG. 1A is a graph of pressure sensor voltage (V) versus time (0.1 sec increments) showing a simple pulse profile (e.g., having same volumes (20 μL), injection rates, duration, and pulse delay (100 ms)) for 6 consecutive injections, labelled Series1, Series2, Series3, Series4, Series5 and Series6.

FIG. 2 is a graph of pressure sensor voltage (V) versus time (0.1 sec increments) showing a second set of a simple pulse profile (e.g., having same volumes (20 μL), injection rates, duration, and pulse delay (100 ms)) for 6 consecutive injections, labelled Series1, Series2, Series3, Series4, Series5 and Series6.

FIG. 3 is a graph of pressure sensor voltage (V) versus time (0.1 sec increments) showing another set of a simple pulsed injection profile for 2000 μL of sample (e.g., having same volumes (100 μL), injection rates, duration, and pulse delay (300 ms)) for 3 consecutive injections, labelled Series1, Series2 and Series3.

FIG. 4 is a graph of pressure sensor voltage (V) versus time (0.1 sec increments) showing another set of a simple pressue pulse profile for multiple injection volumes and injection profiles (e.g., including injection volumes (10 μL, 20 μL, 50 μL, 100 μL, 200 μL, 500 μL, 1000 μL and 2000 μL), volumes, pulse delay, n_pulses, nxt time and max volts).

FIG. 5 is a graph of pressure sensor voltage (V) versus time (0.1 sec increments) showing use of multiple injection profiles to reduce pressure pulse amplitude for various injection volumes, e.g., including 2000 μL with 10 pulses times 20 μL injection per pulse with 100 ms delay between pulses plus 36 pulses times 50 μL injection per pulse with 300 ms delay between pulses; 1000 μL with 10 pulses times 20 μL injection per pulse with 100 ms delay between pulses plus 16 pulses times 50 μL injection per pulse with 300 ms delay between pulses; 500 μL with 10 pulses times 20 μL injection per pulse with 100 ms delay between pulses plus 6 pulses times 50 μL injection per pulse with 300 ms delay between pulses; and 200 μL with 10 pulses times 20 μL injection per pulse with 100 ms delay between pulses.

FIG. 6 is a graph of linearized response/peak max versus time (sec) showing normalized peak profiles for the peak injection profiles of FIG. 5, e.g., including three injection volumes of 200 μL, 500 μL, 1000 μL and 2000 μL with the first injection volume of 2000 μL and the last injection volume of 200 μL indicated and pointed to with suitable lines.

FIG. 7 is a graph of linearized NDIR (counts) versus time (sec) showing: linearized plots, e.g., for 1 ppm, 2 ppm, 5 ppm, and 10 ppm KHP) and a 1 mL injection volume.

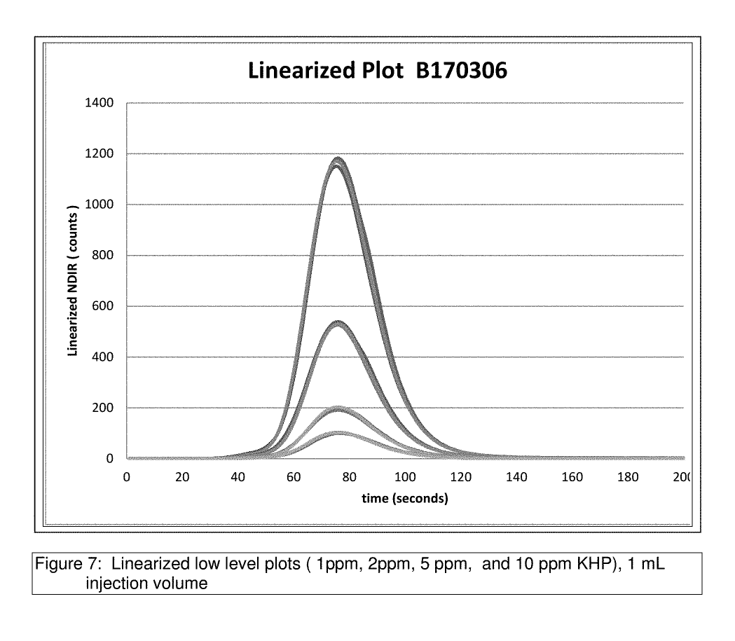

FIG. 8 is a graph of a NDIR response (counts) (normalized to 1000 counts) versus time (sec) showing normalized linear plots, e.g., for 10 ppm, 5 ppm, 2 ppm and 1 ppm).

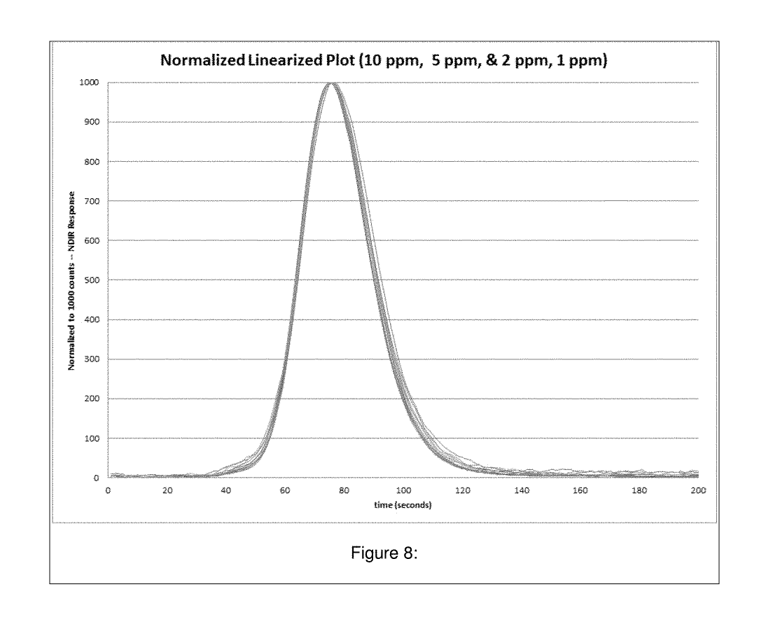

FIG. 9 is a graph of peak area (count-sec) versus injection volumes (μL) showing the measurement of peak area of reagent water as a function of injection volume, and the determination of the slope to permit precise computation of reagent water carbon concentration, e.g., that formed part of a method for determination of a water blank, where y=0.4151x+28.679 and R²=0.9983 and the peak area (count-sec)=0.4151 count-sec/μL*volume (μL)+26.679 count-sec.

FIG. 10 is a graph of peak area (count-sec) versus concentration (ppm, KPH) for a calibration of Total Organic Carbon (TOC), e.g., showing 200 μL injection volume used in determination of blank reagent water computation and a comparison of a computed value with an offset computed value for sensitivity=355.14 count-sec/ppm C, or with mass based sensitivity of S=355.14 count-sec/ppm C/0.200 mL=1775.7 count-sec/μg C.

FIG. 11 is a flowchart showing steps of an OI Analytical—1080 OC stop-flow pulsed-injection method.

FIG. 12 shows a flowchart of a typical TOC injection method,

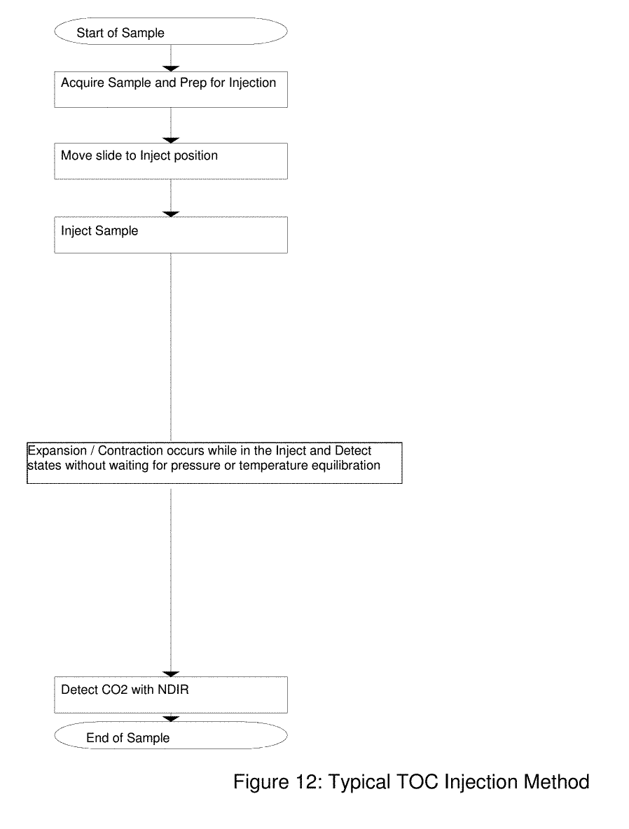

To reduce clutter in the drawing, each Figure in the drawing does not necessarily include every reference label for every element shown therein.

### DETAILED DESCRIPTION OF BEST MODE OF THE INVENTION

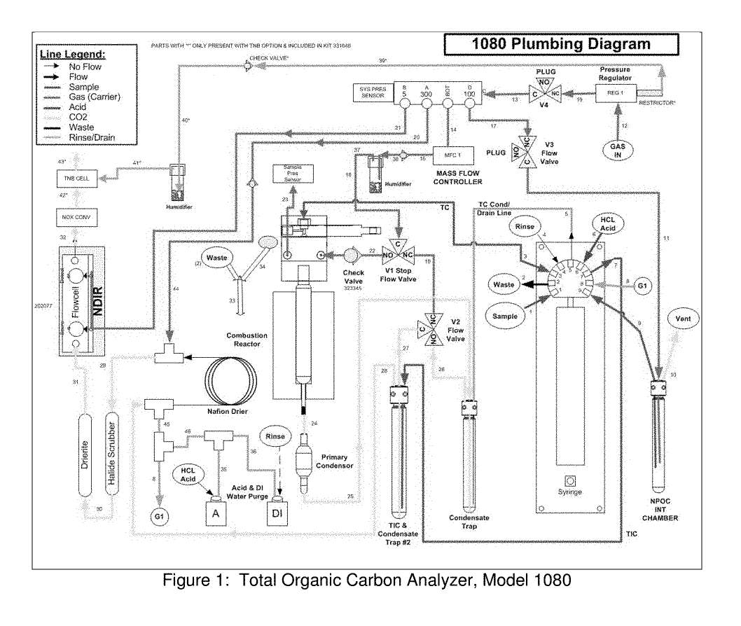

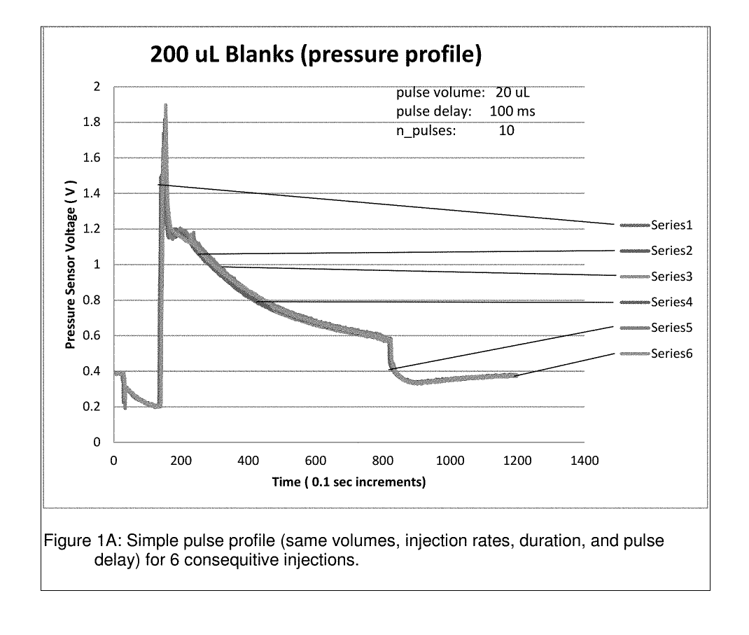

FIG. 1 shows a total organic carbon analyzer/system, e.g., having an injector, a reactor chamber, and a condensation chamber. According to the present invention, by the simple addition of a pair of 3-way valves (e.g., a V1 stop flow valve and a V2 flow valve in FIG. 1), the injector, the reactor chamber, and condensation chamber can be isolated from the rest of the system. The two 3-way valves are set up to allow flow to either pass through the reactor and condensation chamber or bypass it. While in the bypass mode, the sample can be injected at an appropriate rate (or injection profile) so as to allow the sample to condense at or near the same rate that the sample is being injected. Since there is no gas flow, the transport mode across the reactor chamber is primarily due to the steam-generated pressure pulse. Upon passing through the reactor chamber, the steam pulse expands into the condensation chamber and condenses. After allowing time for the reactor to reheat, the system can be set to resume flow of oxygen or air through the reactor and condensate chambers, and then through the remaining TOCA system. Note that this arrangement has the advantage of the reactor-condensation chambers together acting as a ‘trap’, i.e. the sample does not continue to be transported and diffuse with multi-modal dispersion (different dispersion rates in the various sections of the system), but instead is retained within the reactor and condenser volume. A consistent uni-modal peak shape is then detected. Additionally, the transport time to the NDIR as measured from the time the system is switched back to the through flow geometry to the peak start as measured by the NDIR is also very reproducible for a fixed gas flow rate, reactor catalyst packing, and filter set.

The novelty of this design according to the present invention is that the peak shape becomes independent of the amount of sample injected since all the carbon is ‘trapped’ within the reactor-condensation volume. By allowing sufficient time for the reactor to reheat to the initial, pre-sample injected temperature, and having the motive force being solely provided by the gas flow controller, the peak shape is highly Gaussian in profile with tailing due to volumetric constraints of the connecting tubes, flow resistance due to catalyst packing and filters, and detector volumetric time constants that are constant for a given system.

The heat required to convert a fixed volume (or mass) of water (at 20° C.) to steam at 680° C. (the catalytic temperature) is basically the sum of the heat required to raise the temperature of water to 100° C., plus the heat required to vaporize the water to steam at 100° C. (latent heat of water), and finally the heat required to convert the steam from 100° C. to 680° C. The heat required to raise 1 g of water (nominally 1 mL) from 20° C. to 100° C. is given by:

ΔH=Cₚ×ΔT×m

ΔH=4.187 kJ/kg-° C.×(100° C.-20° C.)×0.001 kg

ΔH=0.335 kJ.

The heat required for the phase transition from liquid to steam is given by

ΔH=L_{f}×m/MW

ΔH=40.65 kJ/mole×1 g/18 g/mole

ΔH=2.258 kJ

Finally, the heat required for heating 1 g of steam from 100° C. to 680° C. is given by

ΔH=Cₚ×ΔT×m

ΔH=1.996 kJ/kg-° C.×(680° C.-100° C.)×0.001 kg

ΔH=1.158 kJ

The total energy is therefore 3.751 kJ, of which 60% is due to the vaporization process.

The reactor chamber is a typically a quartz tube, with a sacrificial quartz layer, followed by the catalytic bed, a screen or other filter and has a tapered exit tube. The sacrificial quartz layer has several purposes: it provides a heat reservoir to assist in the vaporization process, it can react with alkali to reduce reaction of alkali with the quartz walls of the reactor tube, and it can absorb the energy of the expansion pulse generated by the injection of room temperature liquids onto a surface at 680° C. The catalytic bed usually consists of highly porous platinum deposited on a ceramic support (e.g. alumina spheres or cylinders). For a ten gram top layer of quartz, the heat capacity is given by:

ΔH=Cₚ×ΔT×m

ΔH=0.733 J/g-° C.×(680° C.−T_{S})×10 g (amount of heat lost in cooling down to T_{S}).

The equilibration temperature of the quartz layer varies depending on how much aqueous sample (volume or mass) is heated by a specific region of the quartz layer. The water or steam can only heat up to the equilibration temperature of the quartz layer. The net result is that pieces of quartz that are struck by droplets of water, rapidly cool and can retain water on their surfaces until sufficient heat is transferred to the water droplet/quartz layer to fully vaporize the water. Sample that is adsorbed on the surface will not combust until the temperature rises sufficiently to vaporize the sample and at the same time have a catalytic surface (downstream of the injection surface) at a high enough temperature with sufficient oxygen present (typically bound to the platinum catalyst) to oxidize the sample to carbon dioxide. For ten grams of quartz (isothermally at 680° C.), and 1 gram of water (at 20° C.), in system at thermal equilibrium, one can compute when the ‘steam’ and quartz would be at the same temperature based solely on heat capacity. To heat 1 g of water to 100° C. requires 335 J. To vaporize the 1 g of water requires 2593 J (2258 joules+335 joules). To heat the steam to the equilibrium temperature T_{S} then requires:

$$\begin{matrix}Δ H ( heating ) = & heating water to 100 ° C . + vaporization + \\ & heating steam to {T}_{S} \\ = & 2593 J + 1.996 J \text{/} g \text{-} ° C . \times ( {T}_{S} - 100 ° C . ) \times 1 g \\ = & 2393 J - 1.996 J \text{/} ° C . \times {T}_{S}\end{matrix}$$

The quartz on the other hand loses energy, so the sign reverses (see above), and

$$\begin{matrix}Δ H = 0.733 J \text{/} g \text{-} ° C . \times ( {T}_{S} - 680 ° C . ) \times 10 g , \\ = 7.33 J \text{/} ° C . \times {T}_{S} - 4984 J .\end{matrix}$$

Solving for T_{S}, 2393 J−1.997 J/° C.×T_{S}+7.33 J/° C.×T_{S}−4985 J=0 (heat lost+heat gained=0)

Or T_{S}=2592 J/(7.33−1.997)J/° C.=486° C.

Since the injected water does not uniformly coat the entire quartz surface, a thermal gradient is created with some of the water being potentially still in the condensed form on the quartz. The conventional system continues to elute the carbon dioxide (reacted sample) as the system is heating the quartz and catalyst back to the control temperature and the water is fully converted to steam.

Similar arguments can be made for the sample striking the same (smaller) section of quartz/catalyst, resulting in even more cooling, and a greater thermal gradient. Most conventional designs direct the sample in a controlled stream so as not to spray the sample on the walls of the reactor. The alkali present in samples will react with the quartz walls, causing them to significantly degrade, eventually losing their structural integrity. A benefit of striking the same region and the generation of a strong thermal gradient is the reduced pressure generated by the vaporization process. At 680° C., water expands 4344 times over its condensed volume: a 50 μL injection of sample expands to 217 mL at 680° C. For a fixed volume of 50 mL (volume above the catalytic bed), this would generate a pressure pulse of 78 psi. In practice, pressures of 20-30 psi are observed. This reduced pressure is primarily due to the actual gas temperature above the catalytic bed being much lower than the catalytic bed, the catalyst/quartz not having sufficient heat capacity to vaporize and heat the steam to 680° C. in the head of the reactor chamber, and is secondarily due to some of the heated gas moving rapidly across the catalytic bed and condensing within a condensation chamber. If too large a volume of sample is injected rapidly onto a conventional reactor bed, the reactor chamber can rupture, releasing pieces of catalyst at 680° C. into the air with likely severe consequences.

For systems that utilize platinum-coated quartz fibers (same mass, much more surface are), the effect is even more severe. Less pressure is generated within the reactor due to slow vaporization of the sample (very low heat capacity of the quartz fibers/wool) which in turn permits injection of large sample volumes (e.g. 1 to 2 mL). The heating of the fibers is largely due to convective heating (the oxidizing gas heats up when it passes through the reactor primarily due to contact with the reactor walls). This effect spreads the combustion of the sample over an even longer duration, making the peak much broader, and decreases the signal to noise ratio of the detector.

The conventional TOCA's peak shape is therefore strongly dependent upon the volume of sample injected, the rate that the sample is injected, and the specific location(s) and materials that the sample is injected onto.

The stopped flow system is designed to have the sample injection rate less than or equal to the condensation rate. This permits variable volume injections that will retain the same peak shape. The stopped flow system still increases in pressure relative to a “no injection” condition, but not to the same extent that a relatively rapid injection into an open system does. Since the system is closed, the resulting combustion product (i.e. carbon dioxide) is not driven by the expansion pulse beyond the condensation chamber, and peak broadening and multi-modal peaks do not occur. This was easily tested by injection of constant masses of carbon by changing both the concentration and volume for the analysis, allowing sufficient time for each volume injected to thermally re-equilibrate before entering the detect mode (i.e. diverting from the bypass mode to oxygen flow through the reactor and condensation chambers).

An alternative method of sample delivery is to pulse the sample into the ‘sealed’ reactor volume. Here the volumetric rate, the duration of that rate, the number of pulses, and the variable delay between pulses is optimized for the specific sample volume being injected. Again, since the sample is ‘trapped’ within the reactor-condensation chamber section of the TOCA, the sample peak shape does not broaden in time due to the slow and delayed injection profile. One advantage is that large volumes of aqueous samples can be injected without generating a large pressure pulse. In practice, small volumes are injected initially, allowing localized cooling of the quartz or catalytic surface, with condensation of the generated steam within the condensate chamber. These small injections are followed by larger volumes until the entire sample volume has been injected. The reactor is then allowed to return to its operational temperature prior to allowing gas flow through the reactor and condensate chamber instead of going through the bypass as previously described. During this time delay, as the reactor heats up, the water present in the reactor continues to vaporize to steam, the steam is transported to the condensate chamber (driven by the pressure generated by the vaporization of the sample), where the steam again condenses. Again the sample is ‘trapped’ within the reactor and condensate volumes, and upon transitioning the valves from the ‘bypass’ mode to the conventional flow through mode, the sample is again oxidized and is transported through the system with conventional detection by the NDIR.

The injection pulse profiles can vary significantly depending upon the desired volume to be injected. In practice, the pulse profile consists of the cumulative effect of multiple pulses. Each pulse consists of a specific injection rate (μL/sec), duration (ms), and delay time (ms) before initiating another pulse. A specific pulse profile may consist of an initial set of pulses, e.g. 10 replicates of 100 μL/sec for 100 ms (or 10 μL injection pulse), with a 100 ms delay, for a total 100 μL injection volume. Alternatively, the 100 μL injection volume can be generated by 5 replicates of 1000 μL/sec for 20 ms, or 20 μL injection pulse with 200 ms delay. This technique gives the analyst considerable flexibility in reducing the resultant pressure pulse and rates of sample introduction relative to the rates of vaporization and condensation within the closed system.

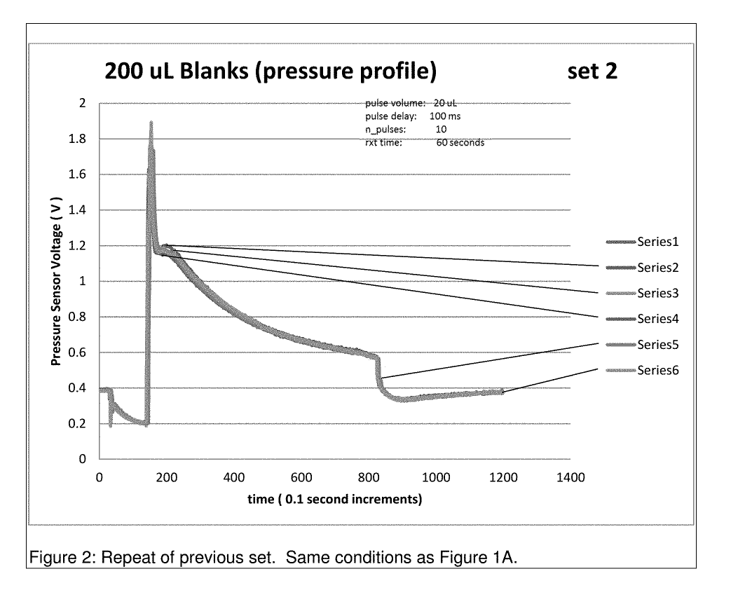

With the addition of a pressure sensor in the injection volume, the pressure profiles can be monitored, allowing the analyst the ability to optimize the injection profile for each desired injection volume. The pressure sensor used has an offset of 0.14 V (ambient pressure), and reads 30.1 psi at 5 V. The pressure data were acquired at 10 Hz. As can be seen in FIG. 1A, using this pulsed injection technique, the pressure profiles generated are extremely reproducible. In this operation, V1 is closed initially for 10 seconds to allow the reactor and condensate chamber to drop to near atmospheric pressure. Next, the sample is injected using 10 pulses consisting of 20 μL per injection with a 100 ms delay between pulses. The decay from 20 seconds to 80 seconds corresponds to the equilibration time required for the reactor to heat up and completely vaporize the water adsorbed by the quartz particles and/or catalytic beads. At 80 seconds, the system was switched from ‘bypass’ mode to conventional flow and the peak was then quantitated. Two sets of 6 replicates each set are shown to illustrate the reproducibility of the injection profile (see FIG. 2).

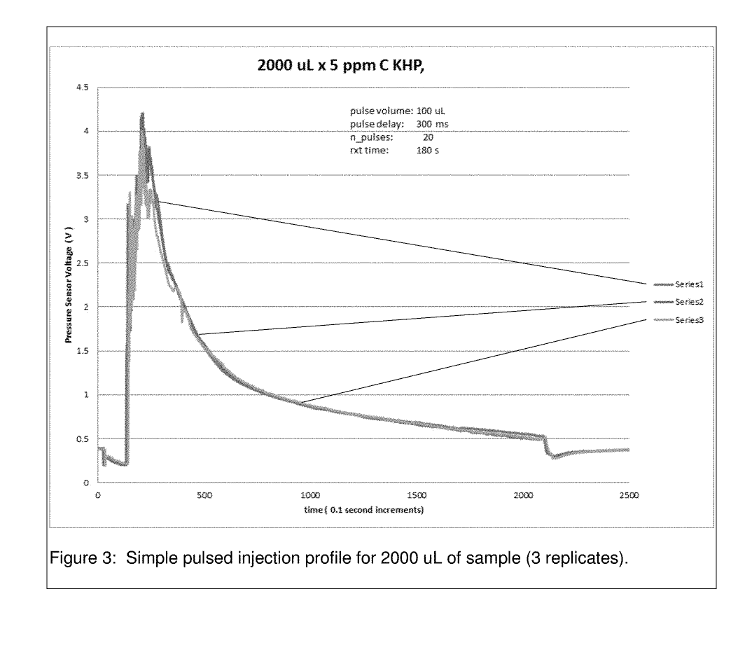

At the other extreme, a 2 mL injection using the pulsed injection technique is shown in FIG. 3. Note again the reproducibility of the injection profile, the peak pressure being only 24 psig, and the longer equilibration time required for the larger total volume injected.

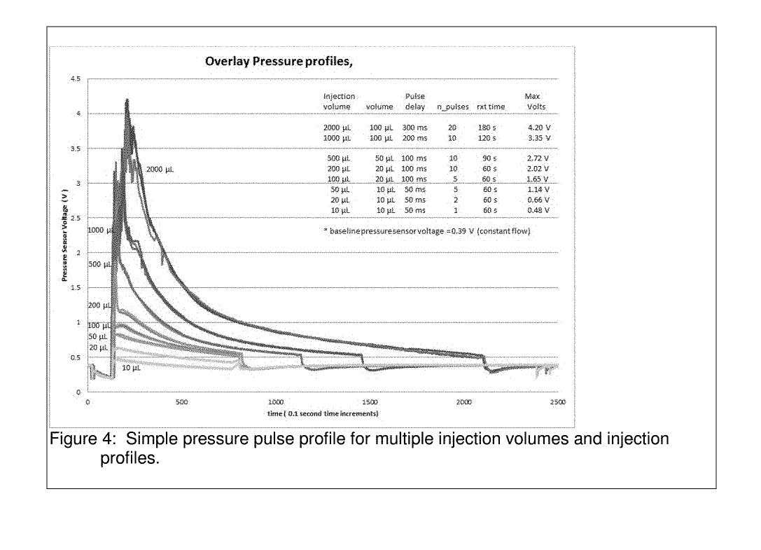

FIG. 4 shows an overlay for a greater range of injection profiles, again showing the reproducibility of the injection profiles, and the trade-off of equilibration time and injection volume.

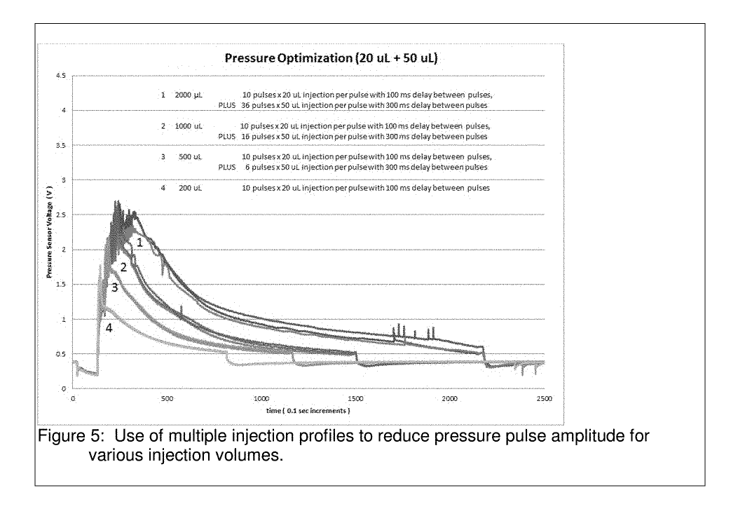

FIG. 5 shows the use of multiple pulse profiles to reduce the pressure pulse even more.

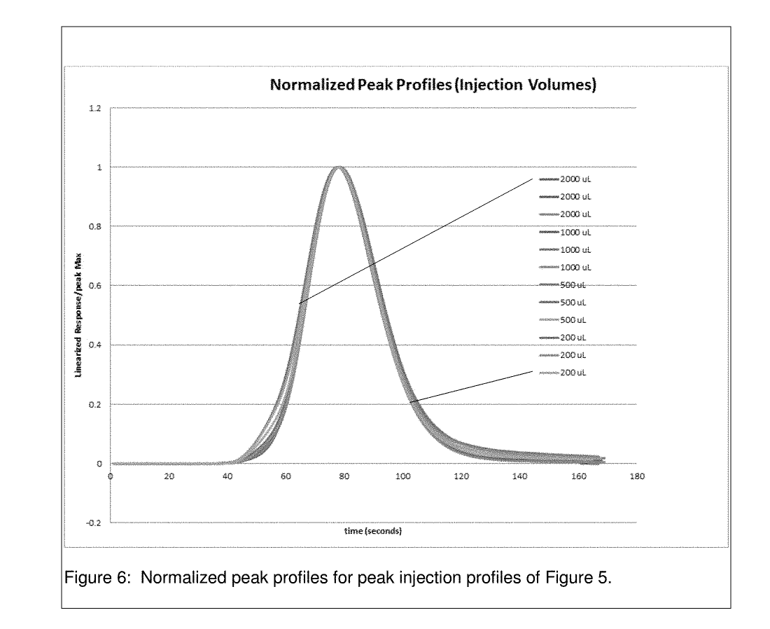

FIG. 6 shows the peak profiles of the pressure profiles of FIG. 5. Even when different volumes are injected (e.g. 2000 μL, 1000 μL, 500 μL, and 200 μL), the peak profiles remain essentially the same uni-modal, near “Gaussian” peak shape.

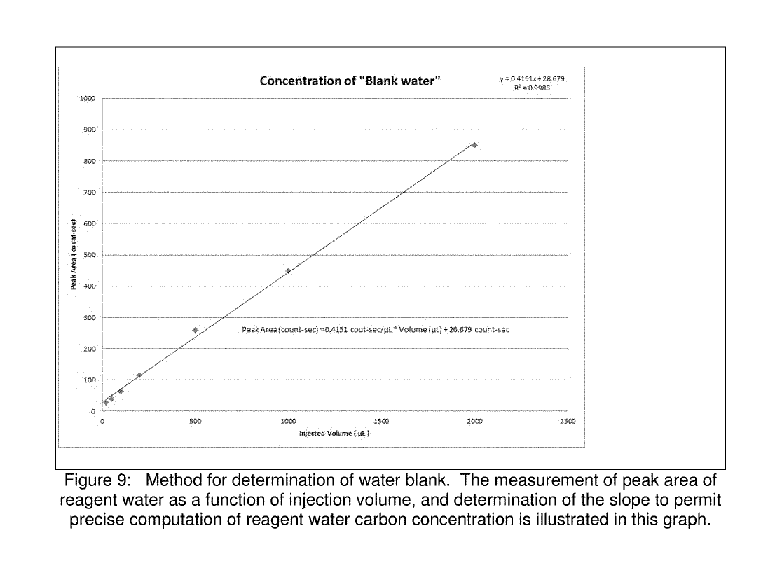

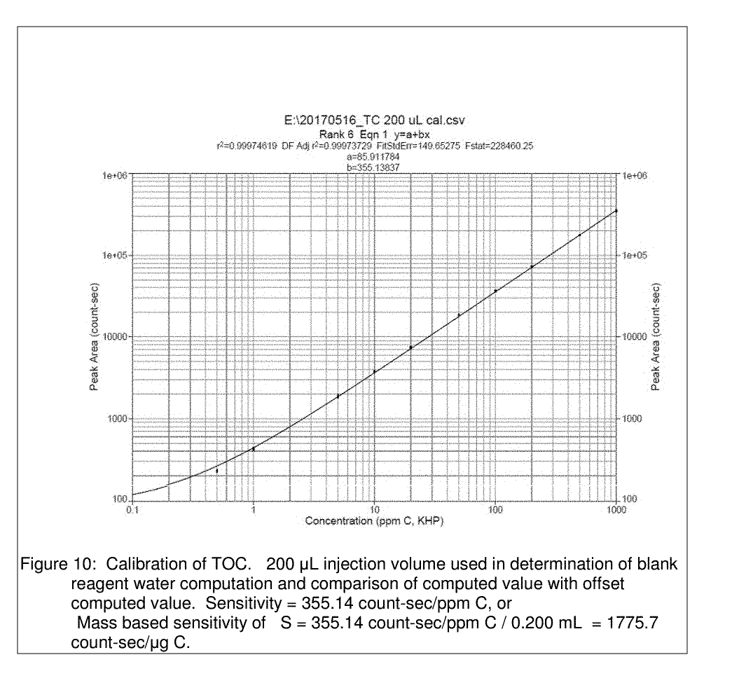

An additional advantage of this invention is the determination of the amount of carbon present in the reagent water. For small injection volumes and low concentrations of carbon in the reagent water, the signal to noise is typically very low, making measurements difficult with a great deal of uncertainty. Since this system can inject large volumes, yet retain the same peak profile, the carbon concentration in the reagent water is readily measured by injecting various volumes of the reagent water and constructing a peak area response versus sample volume as shown in FIG. 9. Fitting the data yields a linear response, with a slope that corresponds to the area per volume being injected. Next, calibration of the system using the standards generated using the reagent water produces another curve as shown in FIG. 10. Fitting this data as peak area versus mass of carbon (concentration of standard*volume) using weighted linear regression to fit the data to a straight line produces a slope (sensitivity measurement in units of peak area per mass of carbon), and an offset (peak area). This form y=m*x+b, (peak area=slope*concentration+offset) can be recast as:

y=m*(x+c),

where c=b/m, and c is the reagent water blank carbon mass within the injection volume.

The reagent water blank concentration is simply the reagent water blank carbon mass divided by the injection volume.

In use, the form is cast so as to determine the concentration from the measured peak area, or:

(x+c)=y/m=y*RRF,

where RRF=1/m and is known as the relative response factor.

The reagent water concentration is therefore easily measured at high sample volumes, but for calibration curves generated using small sample volumes, the uncertainty will increase. Once the RRF is known, then the reagent water concentration is computed by:

[C_{—H20}]=slope(count−sec)/μL*RRF(μg C/(count−sec)).

As shown in FIG. 9, the slope is 0.4151 count-sec/μL, or 415.1 count-sec/mL.

As shown in FIG. 10, the mass sensitivity is 1775.7 count-sec/μg C.

Finally, the reagent water concentration can be determined by:

$$\begin{matrix}[ {C}_{{ }_{—} H 2 O} ] = 415.1 ( count \text{-} sec ) \text{/} mL / 1775.7 count \text{-} sec \text{/} μ g C . \\ = 0.2337 μ g C . \text{/} mL = 233.7 ppb .\end{matrix}$$

This result can be readily compared with that projected by the calibration curve shown in FIG. 10, and is computed as described above by:

$$\begin{matrix}c = 85.91 count \text{-} sec \text{/} 355.1 count \text{-} sec \text{/} ppm C . , or \\ = .242 ppm or 242 ppb .\end{matrix}$$

This offset is primarily due to sorption of carbon dioxide out of the air into the reagent water, and illustrates the problem that exists when trying to make low level standards.

Although not shown, the system also generates ultrapure water that accumulates in the second condensate chamber (downstream from the condensate bulb) due to the TC injection processes. This UHP water can be transferred by means of the syringe into the non-purgeable organic carbon (NPOC) internal sparge chamber, and can be sparged to maintain an inert, carbon free atmosphere. This instrument can also utilize this water to measure UHP blanks, to provide the UHP water to clean and condition the catalyst for low level analyses, and to generate low level calibration standards.

### ALTERNATIVE EMBODIMENTS

By way of further example, in place of the two ‘three way’ valves, a single 4 port valve can be employed to provide isolation of the reactor-condensation chamber section of the TOC from the upstream gas controllers, and from downstream elements, i.e. the associated water removal, halogen scrubbers, particle filters, and carbon dioxide sensor.

In the embodiment disclosed above, the carbon dioxide sensor takes the form of a NDIR. Alternatively, sensors (with their associated support pumps, filters, interfaces, and controllers) may include mass spectrometers, ion conductivity sensors, cavity ring down spectrometers (isotope ratio) specific for carbon dioxide, FTIR spectrometers, and other carbon specific detectors.

In the embodiment disclosed above, the gas flow is regulated by a combination of a pressure regulator and a mass flow controller. The mass flow controller is dependent upon the stability of the upstream pressure provided by the use of a pressure regulator. The pressure regulator is also used in combination with various frits to provide fixed flow rates for the Nafion drier, and sparging of the various reagents, and samples (e.g. sparging of the sample in the removal of volatile organic compounds in the NPOC internal and external modes). Electronic flow control, and/or electronic pressure regulation may be directly substituted for mechanical flow and pressure controllers. Downstream regulation of the flow to the NDIR (as previously utilized in the OIC 1030 TOC analyzer) can also be utilized to ensure constant flow to the NDIR, and additional dilution. Advantages of using the electronic controllers may be offset by their associated higher cost.

In the embodiment disclosed above, a humidifier chamber is used to humidify the oxidizing gas to optimize the catalytic conversion efficiency. The humidifier chamber is a sealed container with an inlet and outlet port and contains water which is sparged by the oxidizing gas. The outlet port of the humidifier chamber has an internal T connection to prevent water (as droplets) from being transported to the reactor. Alternative/y. the method of humidification may include using a Nafion tube immersed in water through which the oxidizing gas (oxygen or air) is passed. The advantage of the Nafion tube may be offset by its cost.

In the embodiment disclosed above, a slider mechanism may be used to divert the injector to either a waste port, or to the center of the reactor tube, e.g., consistent with that disclosed in US 2018/0128547, published 10 May 2018 and corresponding to the aforementioned application Ser. No. 15/807,159, which are both hereby incorporated by reference in its entirety. Actuation of the slider mechanism may include, or take the form of, a mechanical actuator with known stops. The mount for the slider mechanism utilizes a two gland ‘o ring’ seals to seal the mount to the reactor tube. The mount may also utilize a side port to introduce the oxidizing gas into the region immediately above the upper gland seal and effectively sweeps out the otherwise dead volume that would normally be present. The mount also provides a port for an electronic pressure transducer that permits monitoring of the back pressure present in the reactor. As the reactor bed becomes blocked by salts depositing from the sample within the quartz and catalyst beds, the back pressure rises. This back pressure measurement is a convenient monitor for the health of the catalyst and reactor bed, and can be used to indicate leaks in the entire TOC analyzer—requiring only that the user block off the flow prior to the NDIR inlet. Alternate embodiments are to use a diverter valve that sends the sample again to either the waste port or center of the reactor vessel. Additionally, the back pressure can be monitored to signal the system to switch from the bypass mode to the inline mode and initiate the detection and quantitation processes.

In the embodiment disclosed above, a quartz reactor tube is used to contain the platinum catalyst and sacrificial quartz beads and/or inner sacrificial tube, as disclosed in the aforementioned pending application. The quartz bead/chips and/or quartz tube serve to protect the quartz reactor tube from devitrification and decomposition due to reaction with alkali and other metallic compounds potentially present in the sample. The reactor tube utilizes a quartz frit embedded within the tube to support the catalyst and quartz elements and to minimize break down particles of the catalyst and/or quartz elements from migrating into the connecting elements and from there to the condensation chamber, potentially obstructing the gas flow. An alternate design may include to use the same reactor design and quartz wool or platinum screens to provide the same ‘filter’ function as the quartz frit, but at greater risk of physical breakdown of the quartz wool, or much higher cost of the platinum screens. Both the top and bottom of the reactor tube are ground to permit precise sizing and improved resistance to slipping off the connector either the inlet cap or the outlet fitting. It is important not to impede the gas flow by an obstruction, since the design preferentially utilizes the condensation bulb (see below) to extract the heat from the steam and provide a low pressure drop element. If the tubing becomes obstructed, the gas flow slows down and the connecting elements heat up (they drop the heat out). Alternate materials for the reactor tube may be used in place of quartz. These materials need to withstand the pressure, temperature, chemical reactivity, and thermal shock constraints. Examples are reactor tubes fabricated from alumina, titanium oxides, or other high temperature ceramic materials. The reactor itself could be fabricated to be the reservoir for directly containing and heating the quartz and/or catalytic bed, but makes clean up and replacement of the catalyst (after being loaded with non-combustible salts deposited during analysis of salt-containing samples) problematic.

In the embodiment disclosed above, the outlet fitting is a PEEK (polyetheretherketone) fitting utilizing Teflon ferrules. Alternate fittings such as stainless steel (with or without protective coatings, e.g. fluorolon, Teflon, etc.), Teflon unions or a simple piece of Viton tubing may also be used. Stainless steel can be used, but degrades over time due to hydrochloric acid being used in the processing of samples to remove the Total Inorganic Carbon (TIC) content prior to measurement for the Total Organic Carbon (TOC) of the sample. Teflon fittings can also be used, but have a lower operational temperature than PEEK. Viton Tubing can also be used, but typically softens and tends to strongly adhere to the reactor tube making servicing the reactor tube difficult.

In the embodiment disclosed above, a connection between the outlet fitting and the condensate bulb is a PEEK tube. Alternate tubing materials such as Teflon, quartz, stainless, glass-lined tubing, and other inert materials can be used. PEEK is chosen due to inertness, flexibility, and thermal stability and thermal formability.

In the embodiment disclosed above, two condensation elements are utilized. The initial condensate chamber (aka condensation bulb) is used as a vacuum break to prevent the condensed water from being sucked back into the reactor chamber during the equilibration process. The second condensate chamber collects the condensed water and provides a means for blanking the reactor with the ultrapure water (i.e. no TOC content) generated by passage of samples or water blanks through the reactor. In a closed system, the initial steam pressure pulse pushes the gas in the interstitial volumes of the catalyst into the condensate chambers. As the steam is also transported, it condenses in the condensate bulb and condensate chamber. At some point, the pressure in the condensate chamber exceeds that being generated by the sample being vaporized and the flow reverses. The orientation of the condensation bulb is such that the gas within the condensate chamber can return through the condensate bulb without transporting the condensed water back into the reactor. Alternate initial condensate elements may include using coiled tubing consisting of quartz, Teflon, glass lined stainless steel, etc. and are used to provide the initial condensate cooling mechanism, classic water-jacketed condensation elements, and radiator assemblies (finned, convectively cooled devices). A fan is used to convectively cool both condensate elements in the preferred design. The alternate designs typically only cool the initial condensate coil, and have the disadvantage of having water droplets possibly being sucked back into the reactor (dependent upon the injection profiles, and volumes of the initial condensing element). The secondary condensate chamber has the ability to hold the ultra-clean water generated by the previous injections, and provides an additional thermal reservoir to assist in the condensation process as it, and the retained water, are kept cooled to near room temperature by the condensate fan.

In the embodiment disclosed above, a Nafion drier (PermaPure drier) is utilized to drop the dew point of the gas to less than −20° C. When an NDIR is being used, water vapor can interfere with the quantitation of the amount of carbon dioxide in the gas stream. The Nafion drier drops the water vapor pressure to below 0.78 mm Hg. An alternate mode for removal of water is to utilize Peltier coolers at 1-2° C. The Peltier coolers must operate above freezing (0° C.) to prevent blockage of the flow due to ice formation within the Peltier cooler, and thus typically have a water vapor pressure above 4.9 mm Hg. The vapor pressure of water at 20° C. (room temperature) is 17.5 mm Hg. The Nafion drier utilize dry gas at typically 2-3 times the reactor flow in a counter flow design. This spent gas is typically used to sparge the reagent bottles to minimize the vapor pressure of carbon dioxide above the reagent and thereby the carbon dioxide dissolved in the reagent.

In the embodiment disclosed above, a copper shot is utilized to scrub halogen species to prevent reaction within the optical flow path of the NDIR. Alternate materials such as zinc, brass, tin, or other reactive metal particles can also be used.

In the embodiment disclosed above, a final filter of calcium sulfate (aka, Drierite™) is use to further decrease the water vapor to −38° C. (0.121 mm Hg). An additional particulate filter may be used to prevent fine particles and any water droplets from depositing within the NDIR's optical flow path.

In the embodiment disclosed above, the outlet from the NDIR can be coupled to other detectors, such as a Cavity Ring Down Spectrometer, ion conductivity detector, electrolytic conductivity detector for nitric oxide, chemi-luminescence detector for nitrogen or sulfur, and other ancillary carbon dioxide sorption device (traps), gas sampling bags, or cylinders for coupling to mass spectrometers and other systems.

In the embodiment disclosed above, the oxidizing gas is either oxygen or air via pressurized gas cylinders. Gas purifiers for removal of hydrocarbons and carbon dioxide should be used if zero grade air or oxygen is not available. Alternate sources include compressors (with associated driers, filters, pressure reservoir, and regulator), membrane oxygen generators/pumps, and zirconia based high purity oxygen generators.

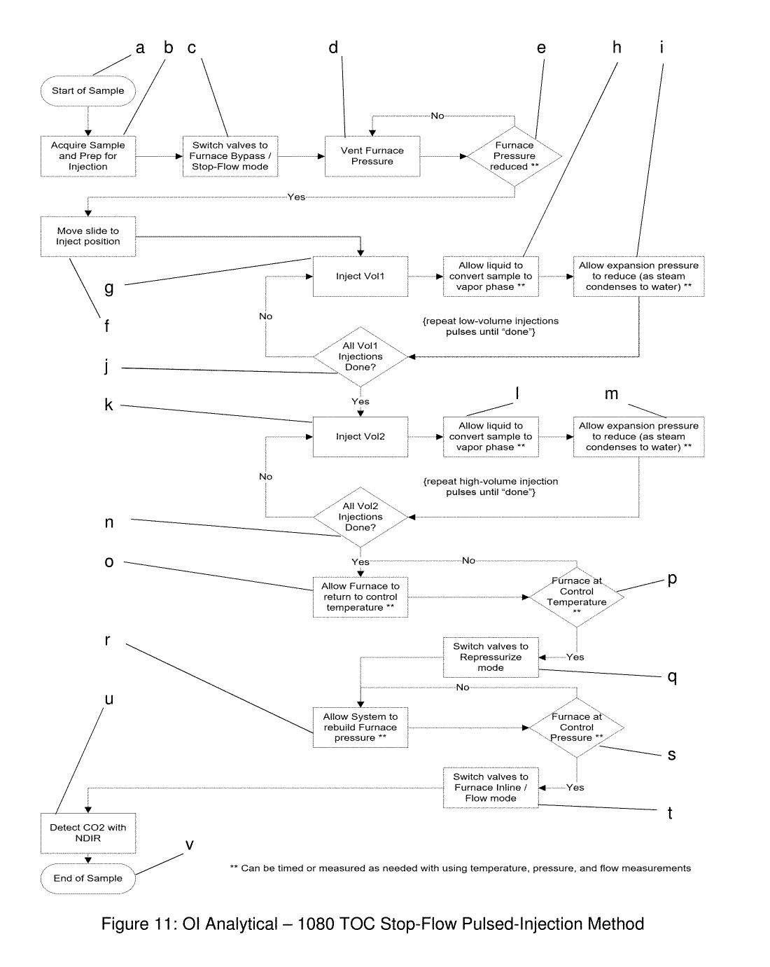

### FIG. 11: The TOC Stop-Flow Pulsed Injection Method

FIG. 11 shows an example of a TOC stop-flow pulsed-injection method by implementing steps a through v. The steps of the TOC stop-flow pulsed-injection method may be implemented in whole or in part by the mass flow controller shown in FIG. 1 in conjunction with one or more other device/components like the valves V1 and V2, a sample injection device/component, the slide valve, the NDIR, a furnace venting device/component, as follows:

In step a, the mass flow controller may be configured to start the sample procedure.

In Step b, the mass flow controller may be configured to acquire the sample and prepare it for injection.

In Step c, the mass flow controller may be configured to switch valves V1 and V2 in FIG. 1 to furnace bypass mode/stop flow mode.

In Step d, the mass flow controller may be configured to vent the furnace/reactor pressure.

In Step e, the mass flow controller may be configured to determine if the furnace pressure is reduced. If not, then the mass flow controller may be configured to repeat step d.

In Step f, the mass flow controller may be configured to move the slide of the valve slide to an inject position.

In Step g, the mass flow controller may be configured to inject volume no. 1. In Step h, the mass flow controller may be configured to allow liquid to convert the sample to a vapor phase, e.g., by repeating low-volume injection pulses until “done”. The conversion step may be timed or measured as needed with using temperature, pressure, and flow measurements.

In Step i, the mass flow controller may be configured to allow expansion pressure to reduce (as steam condenses to water). The expansion step may be timed or measured as needed with using temperature, pressure, and flow measurements.

In Step j, the mass flow controller may be configured to determine if all volume 1 injections are done. If not, then the mass flow controller may be configured to repeat step g.

In Step k, the mass flow controller may be configured to inject volume no. 2.

In Step i, the mass flow controller may be configured to allow liquid to convert the sample to a vapor phase, e.g., by repeating high-volume injection pulses until “done”. The conversion step may be timed or measured as needed with using temperature, pressure, and flow measurements.

In Step m, the mass flow controller may be configured to allow expansion pressure to reduce (as steam condenses to water). The expansion step may be timed or measured as needed with using temperature, pressure, and flow measurements.

In Step n, the mass flow controller may be configured to determine if all volume 2 injections are done. If not, then the mass flow controller may be configured to repeat step k.

In Step o, the mass flow controller may be configured to allow the furnace to return to control temperature. The return step may be timed or measured as needed with using temperature, pressure, and flow measurements.

In Step p, the mass flow controller may be configured to determine if the furnace returned to the control temperature. If not, then the mass flow controller may be configured to repeat step o.

In Step q, the mass flow controller may be configured to switch the valves V1 and V2 in FIG. 1 to repressurize mode.

In Step r, the mass flow controller may be configured to allow the system/analyzer to rebuild the furnace pressure. The rebuild step may be timed or measured as needed with using temperature, pressure, and flow measurements.

In Step s, the mass flow controller may be configured to determine if the furnace is at the control pressure. If not, then the mass flow controller may be configured to repeat step r.

In Step t, the mass flow controller may be configured to switch valves V1 and V2 in FIG. 1 to furnace furnace inline/flow mode.

In Step u, the mass flow controller may be configured to detect CO2 with the NDIR.

In step v, the mass flow controller may be configured to end the sample procedure.

By way of example, the mass flow controller may be configured to implement each step, e.g., by providing suitable control signaling to actuate the various devices/components like the valves V1 and V2, the sample injection device/component, the slide valve, the NDIR, the furnace venting device/component, etc.

4 Port Valve

The four-way valve or four-way cock is known in the art and is a fluid control valve whose body has four ports spaced round the valve chamber and the plug has two passages to connect adjacent ports. By way of example, the plug may be cylindrical or tapered, or a ball. It has two flow positions, and usually a central position where all ports are closed. It can be used to isolate and to simultaneously bypass a sampling cylinder installed on a pressurized water line. It is useful to take a fluid sample without affecting the pressure of a hydraulic system and to avoid degassing (no leak, no gas loss or air entry, no external contamination).

By way of example, the four-way valve may be configured in one position to allow flow between the condensation trap and the reactor, and in another position not to allow flow between the condensation trap and the reactor. The four-way valve may also be configured to allow other gas flow between other devices like the humidifier and the TIC and condensate trap, as well as the reactor and the condensation trap, e.g., consistent with that disclosed herein.

### PATENTS

The following technology is known by the inventors, and summarized as follows:

In U.S. Pat. No. 3,296,435 (by J. L. Teal et al, entitled “Method and apparatus for determining the total carbon content of aqueous streams”, issued Jan. 3, 1967), the basic design of a TOC analyzer is laid out, and the basic tenants of modern TOC instrumentation are discussed. This design requires “the oxygen stream at a predetermined, constant rate of flow” (2-25). The patent also discloses that “a small proportion of highly dispersed carbonaceous material is rapidly injected into the heated zone of the combustion conduit on the upstream side of a diffusing member.” The “diffusion member” is basically chemically inert material (e.g. quartz, sand, alumina, etc.) and/or a catalytic bed (e.g. various reactive metal catalysts, Ni, Fe, Cr, Co, Pt, etc.). Injection volumes are related to the diffusion member's volume and range from 10 to 100 μL, with a maximum of 1 mL due to pressure constraints.

In U.S. Pat. No. 3,530,292 (by H. N. Hill, entitled “Apparatus and method for determination and measurement of carbon in aqueous solutions”, issued Sep. 22, 1970), a total organic carbon analyzer is described that utilizes a sliding plate that serves as an injector into a reaction chamber. Again a constant carrier gas supply is utilized to provide the motive force for transporting the reaction products through a condensation chamber and then to an Infrared Analyzer.

In U.S. Pat. No. 4,352,673 (by Espitalie et al, entitled “Method and device for determining the organic carbon content of a sample”, issued October 1982), the technique uses pyrolysis to differentiate samples of geological sediment at different reaction temperatures within an inert atmosphere and the same sample later in furnace with an oxidizing atmosphere. The system utilizes traps to concentrate each sample, desorb the traps and analyze the carbon dioxide content by NDIR and the organic carbon content by FID.

In U.S. Pat. No. 4,619,902 (by Bernard, entitled “Total Organic Carbon Analyzer”, issued October 1986) the instrument utilizes a digestive chamber for conversion of organic species to carbon dioxide by reaction with persulfate in contact with a catalyst. The system utilizes traps to concentrate the effluent from the digestion chamber since the digestion chamber is continuously purged. After completion of the oxidation phase, the carbon dioxide that was trapped is desorbed and analyzed by an NDIR.

In U.S. Pat. No. 4,968,485 (by Morita, entitled “Arrangements for Preparative Route Leading to Water Analysis”, issued Nov. 6, 1990), a slide block is described that essentially eliminates the accumulation of non-volatile material around the injection port (within the slide assembly) that is mounted onto a combustion chamber. The invention utilizes multiple micro syringe drives to enable automated generation of calibration standards utilizing sample water for the construction of calibration curves.

In U.S. Pat. No. 5,340,542 (by Fabinski et al, entitled “Apparatus for the measurement of the total content of organic carbon and nitrogen in water”, issued Aug. 23, 1994) and in U.S. Pat. No. 5,459,075 (by Fabinski et al, entitled “Apparatus for the measurement of the total content of organic carbon and nitrogen in water”, issued Oct. 17, 1995), the analyzer includes a phase separator, a thermal reactor, a condensation element, and multiple cuvettes for water vapor compensation during measurements of both carbon dioxide and nitrogen oxide (NO) concentrations. The phase separator is used to divert the gas phase (described as the inorganic contribution) from the aqueous phase (described as the organic contribution). The gaseous phase is then dried via a cooler. The aqueous phase is passed through a reactor chamber (combustion), where it is vaporized at 900° C., and oxidized to carbon dioxide. The effluent from the reactor chamber is then dried to the same temperature as the inorganic contribution. The cuvettes containing the organic carbon dioxide and nitrogen oxide (i.e. the inorganic and organic contributions) are switched in and out of the detector assembly and the total inorganic carbon (TIC) and TOC contributions are thus measured.

In U.S. Pat. No. 5,425,919 (by Inoue and Morita, entitled “Total Organic Carbon Analyzer”, issued Jun. 20, 1995) a total organic carbon analyzer (TOCA) is disclosed that utilizes barium hydroxide as a carbon dioxide sorbent during the determination of purgeable organic carbon (POC) in a water sample. In pre-purging the sample with acidification to convert the inorganic carbon to carbon dioxide, the carbon dioxide is purged along with the POC. The barium hydroxide is used to remove the carbon dioxide prior to passing the purge stream so that only the POC passes into the combustion reactor. The TOCA determines non purgeable carbon (NPOC) of the purged sample by combustion. The TOC is simply then the sum of the POC and NPOC measurements. The novelty of the patent is that the barium hydroxide is heated (nominally 30-60° C.) to minimize sorption of the purgeable organic compounds.

In U.S. Pat. No. 5,835,216 (by Y. Koshkinen, entitled “Method of controlling a short-etalon Fabry-Perot Interferometer used in an NDIR measurement apparatus”, issued Nov. 10, 1998), a modification to an NDIR is to reduce the bandpass using an electronically tunable short etalon Fabry_Perot interferometer. This arrangement permits monitoring of specific lines within the bandpass or cutoff wavelength of an optical filter making the NDIR extremely selective with respect to the monitored gaseous species, allowing measurements at multiple wavelengths and reference measurements at non-interfering wavelengths and thus being capable of nearly simultaneous monitoring multiple compounds. This patent discloses how to utilize the cutoff wavelength to ‘calibrate’ the interferometer, and how to use the interferometer as a modulator of the IR radiation allowing use of a DC driven IR source, and thus longer and more stable operation. The long pass filter is typically an interference filter, but can be a specific glass (i.e. Vycor) that has a specific minimum near 4 μm.

In U.S. Pat. No. 6,180,413 (by Ekechukwu, entitled “Low Level TOC Measurement Method”, issued Jan. 30, 2001), TOC is measured by trapping the organic matter in a aqueous sample on a sorbent that is carbon free within a cartridge, then homogenizing the sorbent, inserting a known aliquot (mass) of the sorbent into a furnace, purging with an oxidizing gas to fully combust the sample, with measurement of the resulting effluent containing carbon dioxide to determine the TOC content. The patent discloses that silica gel, alumina, and magnesium silicate are suitable sorbents.

In U.S. Pat. No. 6,375,900 (by M. T. Lee-Alvarez, entitled “Carbon Analyzer with Improved Catalyst”, issued Apr. 23, 2002), a carbon analyzer utilizing a platinum on titania (TiO₂) catalyst is described, in addition to a method for conditioning the catalyst.

In U.S. Pat. No. 6,447,725 (by M. Inoue and Y. Morita, entitled “Total Organic Carbon Meter”, issued Sep. 10, 2002), ultrapure water that is generated by passage of less pure water through the combustion furnace where the removal of organic compounds via high temperature, catalyst assisted oxidation occurs and subsequent capture via condensation of the resulting ‘steam’ into a chamber (water trap). The water from the water trap is then utilized with a test solution to generate low level standards (at or below 50 ppb C), a calibration curve utilizing those standards, to determine the offset ‘blank’ of the reagent water, and a calibration curve for the low level standards.

In U.S. Pat. No. 6,723,565 (by R. J. Davenport and R. D. Godec, entitled “Pulsed-Flow Total Organic Carbon Analyzer”, issued Apr. 20, 2004), TOC in an aqueous stream is determined using a pulsed flow technique with irradiation by UV light and subsequent detection by conductivity measurement in an adjacent chamber. Here the analysis sequence is to fill the analyzer from an aqueous stream, stop the flow, oxidize the sample by UV irradiation until the oxidation process is complete, pulse the oxidized sample into a conductivity meter, and measure the conductivity. Multiple conductivity measurements (both prior to the irradiation cell and after the irradiation cell, and with and without the UV irradiation) are required to determine the TOC content for low level samples. The use of catalytic electrodes being exposed to the UV light (generation of peroxide, with light cleavage to hydroxyl radicals for the oxidation of organic species) is also disclosed.

In U.S. Pat. No. 6,793,889 (by U. W. Naatz et al, entitled “Wide-range TOC instrument using Plasma Oxidation”, issued Sep. 21, 2004), the aqueous or gaseous sample and an oxidant gas is contained within a chamber that is exposed to the plasma via a window transparent to the plasma with detection of the CO2 produced by FTIR, NDIR, or ion conductivity measurements. Here a barrier dielectric discharge (aka atmospheric glow discharge, or silent discharge) is used to provide the energy required to form highly reactive species (ozone, excited oxygen, oxygen atoms, peroxides and especially hydroxyl radicals from the dissociation of peroxides formed by the reaction of energetic electronically excited species and high energy photons with water) that react with the carbonaceous material in the sample to form carbon dioxide.

In U.S. Pat. No. 8,114,676 (by G. B. Conway, et al, entitled “Carbon Measurements in Aqueous Samples using Oxidation at Elevated Temperatures and Pressures”, issued Feb. 14, 2012)* the sample is cool upon introduction into a high pressure and high temperature vessel. The vessel is then heated to supercritical fluid (water) conditions (above 374° C., and 22.12 MPa) that result in the rapid and complete oxidation of carbon within the aqueous sample. The vessel is then cooled to (or near) room temperature where the carbon dioxide is purged from the reactor and measured. (*See also U.S. Pat. Nos. 8,101,418, 8,101,419, and 8,101,420.)

In U.S. Pat. No. 9,194,850 (by S. Inoue and K. Noto, entitled “Measurement Device for Total Organic Carbon”, issued Nov. 24, 2015), the problem to be solved is the difference in flow rates when the TIC sparging within the syringe (150 mL/min) is different than the flow rate is when the TOC is not sparging (230 mL/min) This generates a change in the baseline level that can affect the measurement accuracy due to distortion of the peak and difficulty in determination of the start of the CO2 peak being detected by the NDIR. This patent discloses a method that automatically adjusts an electronic valve to maintain a constant make-up flow when the system is in the TIC sparging state.

The OI Analytical 1030 TOC utilizes electronic flow controls and mass flow meters to allow the user to maintain an adjustable constant flow to the NDIR. In the OI design, combining the effluent from either the TIC sparge or TOC reactor to a secondary flow with a secondary flow allowed dilution of high level standards to extend the dynamic range of the TOC system. Similarly, the 1030 Solids module utilizes a gas dilution module for incorporation with a cavity ring down spectrometer (CRDS) for isotopic quantitation of carbon, again monitoring the gas supply flow rate and controlling the system flow to generate a specific gas flow rate and sample dilution to the CRDS.

All of these aforementioned US patents set forth above are incorporated by reference herein.

### Line Legend in FIG. 1

FIG. 1 includes a line legend showing lines associated with fluid flows, e.g., including no flow (e.g., see line no. 4), flow (e.g., see line no. 2), sample (e.g., see line nos. 1, 3, 7 and 8), gas (Carrier) (e.g., see line nos. 11-23, 37-38 and 44), acid (e.g., see line no. 6), CO2 (e.g., see line nos. 10, 24-32 and 42-43), water (e.g., see line no. 2) and rinse water (e.g., see line no. 4).

### THE SCOPE OF THE INVENTION

While the invention has been described with reference to an exemplary embodiment, it will be understood by those skilled in the art that various changes may be made and equivalents may be substituted for elements thereof without departing from the scope of the invention. In addition, may modifications may be made to adapt a particular situation or material to the teachings of the invention without departing from the essential scope thereof. Therefore, it is intended that the invention not be limited to the particular embodiment(s) disclosed herein as the best mode contemplated for carrying out this invention.

What is claimed is:

1. A total organic carbon analyzer, comprising:
an injector configured to provide a sample;
a reactor configured to vaporize the sample received;
condensation components configured to condense and trap the sample vaporized by the reactor; and
two three-way valves arranged between the reactor and the condensation components and configured to allow flow to either bypass or pass through the reactor and the condensation components, while in the bypass mode, the sample being injected at an appropriate rate so as to allow the sample to condense at or near the same rate as the sample is being injected.

2. A total organic carbon analyzer according to claim 1, wherein the two three-way valves comprise:
a stop flow valve V1 having a port C, a normally open port NO and a normally closed port NC; and
a flow valve V2 having a corresponding port C, a corresponding normally open port NO and a normally closed port NC.

3. A total organic carbon analyzer according to claim 2, wherein
the condensation components comprise a condensate trap and a total inorganic carbon (TIC) and condensate trap;
the normally open port NO of the stop flow valve V1 is coupled to a port of the reactor;
the normally closed port NC of the stop flow valve V1 is coupled to the corresponding normally closed port of the flow valve V2;
the corresponding normally opening port NO of the flow valve V2 is coupled to and receives condensate trap CO2 gas from the condensate trap; and
the corresponding port C of the flow valve V2 is coupled to the TIC and condensate trap to provide the condensate trap CO2 gas from the condensate trap to the TIC and condensate trap.

4. A total organic carbon analyzer according to claim 3, wherein the condensation components comprise a primary condenser coupled between the reactor and the condensate trap and configured to receive reactor CO2 gas from the reactor and provide primary condenser CO2 gas to the condensate trap.

5. A total organic carbon analyzer according to claim 3, wherein the TIC and condensate trap is configured to receive the sample.

6. A total organic carbon analyzer according to claim 3, wherein the total organic carbon analyzer comprises a check valve coupled between the normally open port NO of the stop flow valve V1 and the port of the reactor.

7. A total organic carbon analyzer according to claim 3, wherein the total organic carbon analyzer comprises a humidifier coupled to provide humidifier gas to the port C of the stop flow valve V1.

8. A total organic carbon analyzer according to claim 3, wherein the total organic carbon analyzer comprises a nafion tube immersed in water and coupled to provide humidifier gas to the port C of the stop flow valve V1.

9. A total organic carbon analyzer according to claim 3, wherein the total organic carbon analyzer comprises a non-dispersive infra-red detector (NDIR) configured to receive the TIC and condensate CO2 gas, detect of the carbon dioxide contained therein and provide NDIR signaling containing information about the same.

10. A total organic carbon analyzer according to claim 3, wherein the total organic carbon analyzer comprises a mass spectrometer, an ion conductivity sensor, a cavity ring down spectrometer (isotope ratio) that is specific for carbon dioxide, or a Fourier-transform infrared (FTIR) spectrometers.

11. A total organic carbon analyzer according to claim 3, wherein the total organic carbon analyzer comprises a combination of a pressure regulator and a mass flow controller configured to regulate gas flow.

12. A total organic carbon analyzer according to claim 3, wherein the total organic carbon analyzer comprises an electronic flow control, and/or electronic pressure regulation configured to regulate gas flow.

13. A total organic carbon analyzer, comprising:
an injector configured to provide a sample;
a reactor configured to vaporize the sample received;
condensation components configured to condense and trap the sample vaporized by the reactor; and
a multi-way valve arrangement arranged between the reactor and the condensation components and configured to allow flow to either bypass or pass through the reactor and the condensation components, while in the bypass mode, the sample being injected at an appropriate rate so as to allow the sample to condense at or near the same rate as the sample is being injected.

14. A total organic carbon analyzer according to claim 13, wherein the multi-way valve arrangement comprises two three-way valves that includes:
a stop flow valve V1 having a port C, a normally open port NO and a normally closed port NC; and
a flow valve V2 having a corresponding port C, a corresponding normally open port NO and a normally closed port NC.

15. A total organic carbon analyzer according to claim 14, wherein the multi-way valve arrangement comprises a single 4 port valve.
